(19) United States
(12) Patent Application Publication
(10) Pub. No.: US 2006/0153330 A1
(43) Pub. Date: Jul. 13, 2006

(54) System for radiation imaging and therapy of small animals

(76) Inventors: John W. Wong, Bloomfield Hills, MI (US); Elwood P. Armour, Bloomfield Hills, MI (US)

Correspondence Address: BRINKS HOFER GILSON & LIONE, P.O. BOX 10395, CHICAGO, IL, 60610, US

(21) Appl. No.: 11/205,701

(22) Filed: Aug. 17, 2005

Related U.S. Application Data
(60) Provisional application No. 60/603,103, filed on Aug. 19, 2004.

(51) Int. Cl. A61N 5/10

(52) U.S. Cl. 378/65

(57) ABSTRACT

A system is equipped with a computer-controlled rotating gantry and includes at least one radiation source mounted to the gantry. The radiation source moves along an arcuate path as the gantry rotates and emits an x-ray beam at a first energy level for imaging of a small animal and emits an x-ray beam at a second energy level for radiation therapy of the small animal. The first energy level is less than the second energy level. The system's imaging capabilities are implemented with the use of a flat panel detector.

### RELATED APPLICATION

This application claims the benefit of U.S. Provisional Application No. 60/603,103 filed Aug. 19, 2004, the entire contents of which are incorporated herein by reference.

### BACKGROUND

The present invention generally relates to radiation imaging and therapy for small animals.

In cancer research, small animals, such as mice, rats, and rabbits are used extensively to evaluate the effectiveness of novel treatment as well as treatment related toxicity. In combination with advanced imaging methods, small animal research allows detailed study of biological processes, disease progression, and response to therapy, with the potential to provide a natural bridge to the clinical environment and contribute substantially to the development of human medicine. These studies hold promise of saving years and significant cost of human clinical trials.

At present, small animal imaging initiatives have focused primarily on the development of systemic treatment methods and biomarkers to detect tumors and monitor or predict responses. Little attention has been directed at advancing the treatment of cancer with radiation. This is ironic and unfortunate, as radiation therapy is a major and effective treatment modality that explicitly exploits imaging methods for target definition and treatment delivery. Instead, over the past couple of decades, there has been a widening technological disparity between laboratory radiation research and clinical radiation therapy; the latter very much championed by the industry. Whereas simple single beam/single fraction techniques are commonly used to irradiate laboratory animals, advanced three-dimensional (3D) and computer-controlled delivery technologies are now used clinically to pinpoint fractionated conformal radiation therapy (CRT) and intensity modulated radiation therapy (IMRT). The uniform dose distributions delivered to research animals bear little resemblance to the highly non-uniform dose distributions that conform tightly to the human tumor targets, while sparing surrounding critical structures. Traditional understanding of tumor control probability (TCP) and normal tissue complication probability (NTCP) based on animal radiation research has become increasingly less suitable to model response and predict outcome of modern radiation therapy. The technological disparity also presents a difficult hurdle in the development of novel treatment methods that combine conformal irradiation and other therapeutic agents, such as drugs, biomarkers, angiogenic modifiers and inducible gene products. There, the temporal, functional and anatomic characteristics of the therapeutic synergism and toxicity must be quantified, but would be difficult to assess using research animals given the different irradiation methods between the laboratory and the clinic.

High resolution imaging, dose planning and conformal irradiation capabilities are presently unavailable for small animal research despite their need in the era of anatomical and biological image guided radiation therapy. In retrospect, small animal radiation research has needed an injection of new imaging and irradiation technologies for some time now. While early small animal radiation research established many of the fundamentals for human treatment, the methods of irradiation lag far behind those used in the modern clinical setting. Important questions such as normal tissue toxicity due to non-uniform dose distributions cannot be adequately studied with current animal radiation methods.

There is a need to conduct animal radiation research that bears realistic resemblance to human treatment to improve our understanding of tumor and normal tissue response to non-uniform irradiation.

### SUMMARY

In satisfying the above need, as well as overcoming the enumerated drawbacks and other limitations of the related art, the present invention provides a system for small animal radiation imaging and therapy.

The system integrates imaging and radiation delivery, and, optionally, treatment planning capabilities. The system is equipped with a computer-controlled rotating gantry and includes at least one radiation source mounted to the gantry. The radiation source moves along an arcuate path as the gantry rotates and emits an x-ray beam at a first energy level for imaging of a small animal and emits an x-ray beam at a second energy level for radiation therapy of the small animal. The first energy level is less than the second energy level. The system's imaging capabilities are implemented with the use of a flat panel detector, for example, an a:Si flat panel detector.

In some embodiments, three kilovoltage (kV) x-ray sources are mounted to the gantry to provide combination-imaging and radiation functions. The sources emit radiation with an energy in the range between about 100 kVp and 250 kVp. On-board cone-beam CT (at, for example, ≦0.5 mm resolution) may be implemented along with the flat-panel detector for imaging capabilities. In certain embodiments, remountable x-ray focusing lens are deployed on each radiation source to achieve “dose painting” at —1 mm resolution.

In particular embodiments, kV dose calculations based on Monte Carlo and pencil beam convolution methods are coupled with a clinical 3D planning system to facilitate conformal irradiation experiments on the system. Accuracy of the dose calculations can be verified at 1 mm resolution using a water-equivalent 3D gel dosimetry system and 2D radiochromic film in well-defined phantom arrangements.

Other features and advantages will become apparent from the following description, and from the claims.

### BRIEF DESCRIPTION OF THE DRAWINGS

FIG. 1a is a perspective view of a system for small animal radiation imaging and therapy in accordance with an embodiment of the invention;

FIG. 1b is an end view of the system;

FIG. 1c is a close-view of an x-ray source in accordance with an embodiment of the invention;

FIG. 2 depicts the dose painting capability of a pivoting x-ray source;

FIG. 3a is a schematic of x-ray diffraction from a crystal lattice;

FIG. 3b is a schematic of a multi-layer lens with varying curvature and varying layer separation;

FIG. 4 is a schematic of an x-ray lens for the system of FIG. 1 in accordance with an embodiment of the invention;

FIG. 5 is a schematic of an alternative x-ray lens for the system of FIG. 1 in accordance with an embodiment of the invention;

FIG. 6 is a graph showing the intensity of three focused x-ray beams; and

FIG. 7 shows EGS4 simulations of 120 Kev mono-energetic x-ray irradiating the head of a rabbit.

### DETAILED DESCRIPTION

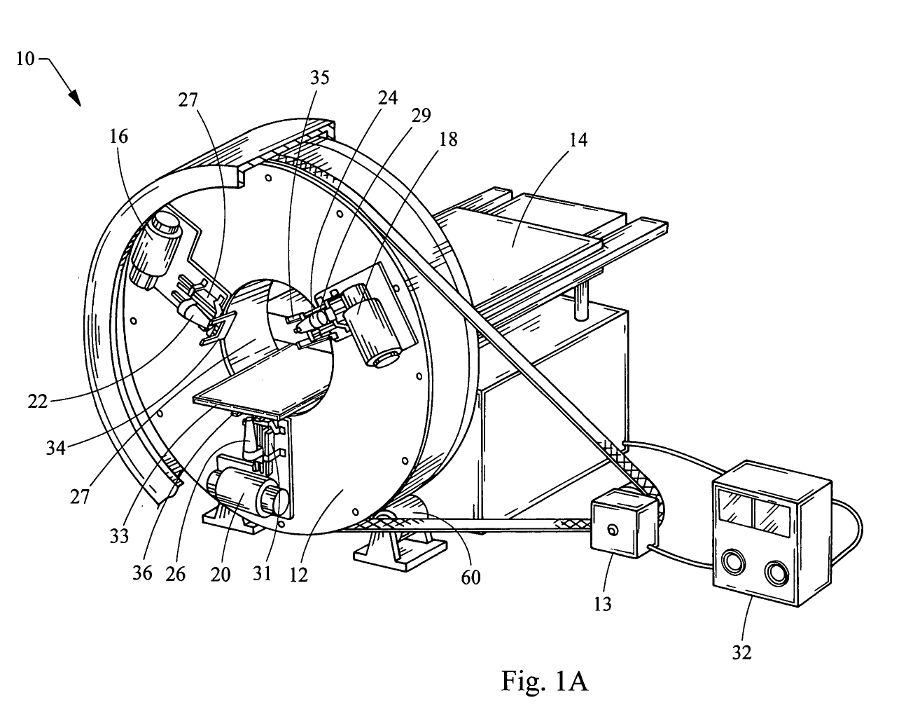

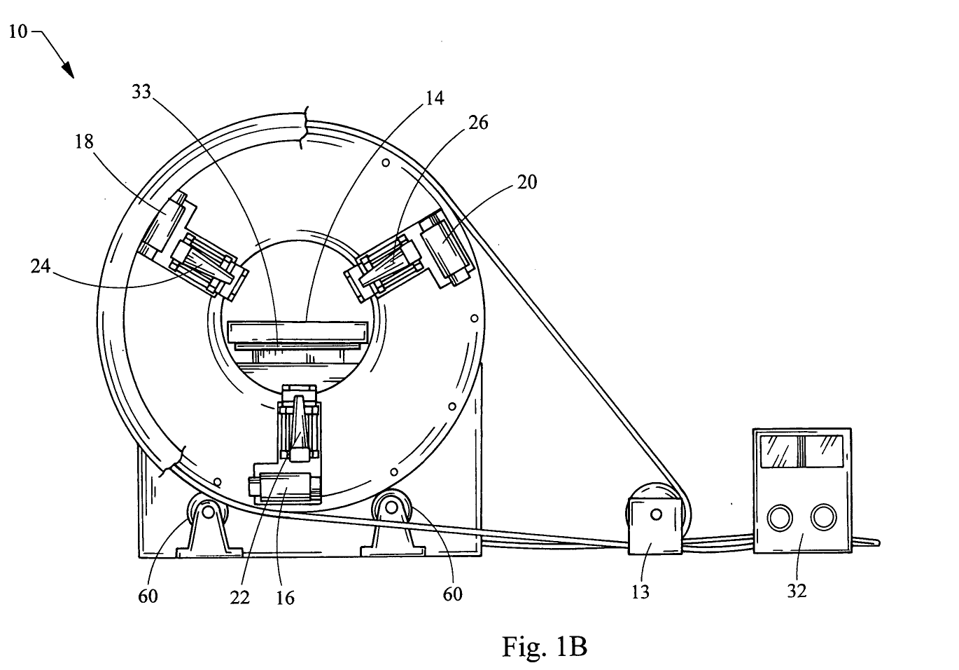

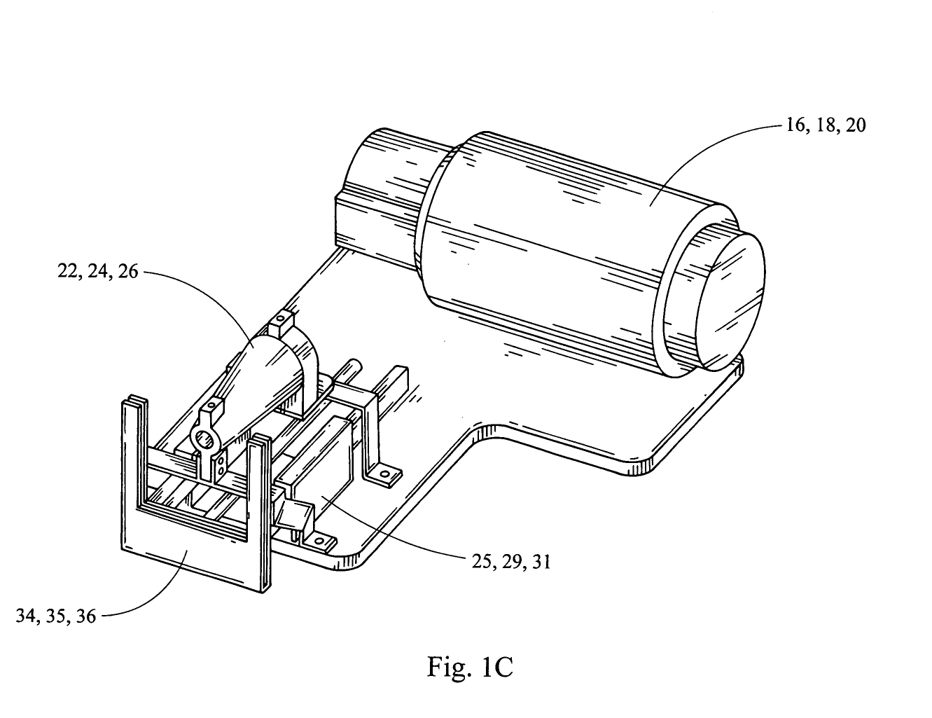

Referring to FIGS. 1a, 1b, and 1c, in accordance with the invention, an image-guided small animal radiation system 10 facilitates accurate and efficient delivery of dose distributions in small animals. The system 10 includes an isocentric gantry 12 and a horizontal animal support couch 14. The system 10 also includes three x-ray sources 16, 18, 20, and each source is fitted with interchangeable x-ray optics 22, 24, 26 and a respective physical collimation system. The three x-ray sources 16, 18, 20, in some implementations, are constant voltage x-ray sources, such as kilovoltage sources that provide a combined dose rate of ≈200 cGy/min per source. Three dimensional treatment planning, dose calculations and dose verification capabilities ensure high quality dosimetry data for analysis. On-board radiographic and cone-beam CT imaging ensure high precision irradiation.

The gantry includes an aperture 27 to accommodate animals ranging from mice to small dogs, and to facilitate treatment of animals and research with laboratory and spontaneous tumor models. The anatomical CT images of the animal in treatment position facilitate accurate registration with biological images acquired with other modalities, such as PET, MRI and MRSI. In turn, in-depth in vivo mechanistic studies of tumor control and normal tissue toxicity, supported by ex vivo validation, can be conducted for different image guidance strategies. The system 10 can obtain CT voxel resolution of ˜0.5 mm with 1 cGy imaging dose, localized radiation dose at a FWHM of ˜1.5 mm and a dose rate of ˜200 cGy per min.

The gantry 12 is a vertically anchored, isocentric, (375°) rotating gantry rotated by a belt-drive motor 13. The aperture 27 is about 45 to 50 cm in diameter, and the source to isocenter distance is about 25 cm. A computerized system 32 with appropriate user-interface and database implemented to control all high precision mechanical motions of the components of the system 10, and to allow convenient repositioning for repeat study.

The gantry 12 with isocentric rotation achieves a high geometric accuracy for irradiation. The horizontal couch 14 supports the animal in its natural prone position, thus most stable setup.

The x-ray sources 16, 18, 20 can be dual focus (0.4 mm and 1.5 mm) Comet x-ray sources with collimators, at 150 lbs each, which are positioned at 25 cm from the isocenter, at equal angular separation. Each x-ray source is fixed to the gantry 12 to maintain mechanical rigidity for accurate gantry rotation. For simplicity of operation, each of the three dual focus x-ray sources 16, 18, 20 is powered by its own generator to produce x-rays with energies up to 250 kVp. These generators can be operated continuously at high output to maximize dose rate. A 250 kVp incident beam when adapted with an x-ray lens (described below) increases the number of focused photons with energies between 40 and 80 keV. For irradiation experiments involving larger animals, the 250 kVp beams can be appropriately hardened with filters to minimize the attenuation of the larger bony structures due to photoelectric effects.

One of the x-ray sources can serve the dual purpose of imaging and dose delivery. For example, a lower energy x-ray beam (˜120 kVp) can be used for imaging. Because the 250 kVp generator produces continuous output, a fast mechanical metal 100 kVp x-ray shutter may be installed to allow frame-by-frame exposure for cone-beam CT imaging.

Projection images are acquired with a detector 33. For example, the detector 33 can be a 20.5 cm by 20.5 cm (512 by 512 elements) flat-panel a:Si detector placed at ˜25 cm from the isocenter, orthogonal to the axis of the x-ray source. With an image magnification factor of about two for an object placed at the isocenter, the system 10 can accommodate an animal with a 10 cm diameter torso. Given the finite size of the 0.4 mm focal spot of the Comet tube, this particular geometry also provides cone-beam voxel resolution of ˜0.5 mm. Even though the quality of cone-beam CT imaged is affected by the magnitude of x-ray scatter, the degradation in image quality, however, is minor, or even insignificant, for the small animals intended for the system 10. In the event that scatter corrections are needed, one or several effective solutions can be implemented, such as modest increasing in dose, or the use of a scatter rejection grid.

The flat panel detector 33 can be read out at a rate of five projections per second. The mechanical shutter operates at the same frequency, but with a shorter pulse (time) width, to ensure complete acquisition of each projection. The operation of the shutter and the panel readout are synchronized.

At five frames per second, a typical set of 300 projection images for cone-beam CT can be acquired in about 60 seconds. A cone-beam reconstruction software implemented in the computer system 32 allows non-uniform projection angular increments such that the rotation of the gantry 12 does not have to be tightly synchronized with image capture. The reconstruction of 256×256×256 voxels CT data can be achieved in less than 2 min.

In order to achieve ≦0.5 mm CT voxel resolution, the center of rotation of the system 10, with its component weight (motors, cabling and cooling) of about 900 lbs, does not wander by more than ±1.0 mm. Further, the movement of the center is reproducible with rotation and can be corrected to within 0.1 mm. For normal gantry motion for treatment setup, the gantry 12 rotates at speeds up to 0.1 revolution per sec.

The geometry of cone beam CT imaging is suited for use with the three x-ray source arrangement 16, 18, 20 to achieve a dose rate of ˜200 cGy/min for irradiation. The three-source arrangement also lends itself naturally to weight balancing on the rotating gantry. When the beam aperture is larger than 4 mm×4 mm, the larger 1.5 mm focal spot x-ray source can be used, as physical collimation has minimal effect on radiation output. With the larger 1.5 mm focal spot, the Comet x-ray tube can be run continuously at a current of about 7 mA. Using the general exposure rule of 10 mR per mAs at 1 meter, one Comet tube can provide about 1120 mR/sec, ˜67 cGy/min at 25 cm. Therefore, 3 x-ray sources combine to deliver a dose rate of about 200 cGy/min at the isocenter of the system 10. The isocentric 3-source arrangement provides significant advantages in terms of high throughput and precision over the use of a single source that would require significant animal or source adjustment for conformal irradiation. In some embodiments, one or two of the sources may not be used, resulting in modest but acceptable increase in treatment time.

The sources 16, 18, and 20 are provided with adjustable beam shaper mounts 34, 35, and 36. For the larger field applications with a radius >4 mm, plastic, aluminum or lead collimators and modifiers can be used for field shaping and intensity modulation. For example, multileaf or multirod collimators can be used. Or rods manually placed in Styrofoam may be used to shape the beam. These devices are positioned appropriately to minimize beam penumbra.

For field sizes <4 mm where dose outputs can drastically be reduced with physical collimation, each lens or optics 22, 24, 26 is a 40-80 keV lens employed to produce a pencil beam of ˜1.5 mm FWHM. The working condition of the lens is adjusted to fit the 25 cm isocenter of the system 10. Docking mechanisms 25, 29, 31 are implemented to allow efficient but robust attachment or detachment of respective lens 22, 24, 26 to the respective x-ray source 16, 18, 20. The smaller 0.4 mm focal source is used for x-ray generation in order to minimize widening of the emitted focused pencil beam due to source blurring. Conservatively, a diagnostic x-ray source with a 0.4 mm focal spot can be run continuously at 2.8 mA. With the 3× lens replacement gain factor, one x-ray source/lens combination can achieve ˜80 cGy/min output at 25 cm. The three x-ray source/lens arrangement ensures a dose rate ˜240 cGy/min. Use of 250 kVp x-rays also increases dose rate. The 40-80 keV focused pencil beams is advantageous for biological image guided “dose painting” applications, or in the study of the biological changes in tumor-normal tissue microenvironment in mice and other superficial targets where enhanced bone attenuation is not significant.

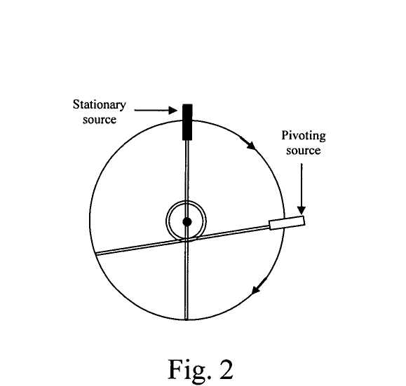

The three focused pencil beams intersect at the isocenter from any gantry orientation. Dose painting is accomplished by irradiation at different couch and gantry positions. With the system 10, an additional capability of pivoting each pencil beam under computer control in the plane of gantry rotation is implemented. FIG. 2 shows that a pivoting source greatly enhances the dose painting capability of a lens versus a stationary source.

The degree of pivoting of each x-ray source 16, 18, 20 is limited in order to maintain the integrity of isocenter accuracy. In addition to x-ray source pivot, a computer controlled rotating stage is deployed on top of the horizontal couch 14 to support the non-coplanar irradiation of the animal with or without lens. The rotating stage greatly enhances the capability of conformal irradiation using the system 10. For simplicity of operation, the rotating stage is a separate “add-on” apparatus independent of the couch. With an aperture of about 45 to 50 cm, the system 10 can accommodate a substantial rotating stage, and in turn, a wide range of animal sizes. All animal support mechanisms of the system 10, including the couch top, can be constructed with minimally attenuating materials, such as carbon fiber and Styrofoam.

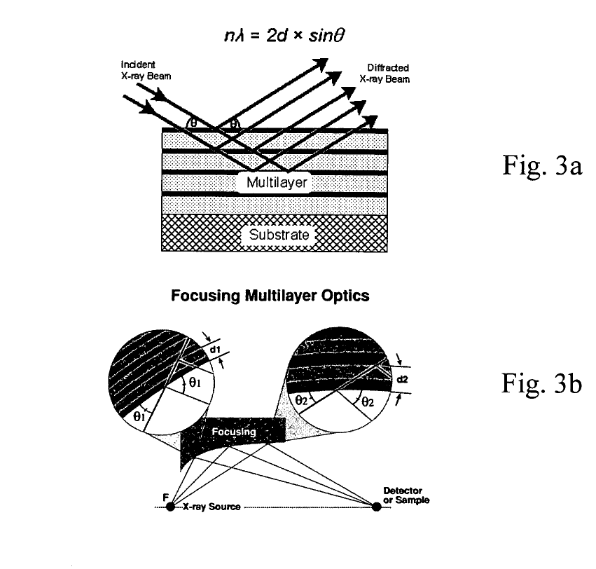

In order to maintain adequate dose output for a beam localized to <4 mm, x-ray focusing lens are employed. X-ray lenses are based on the principles of x-ray diffraction from the crystal lattice of specific materials. A particular curved multi-layer crystal x-ray lenses is produced by Osmic, Inc., Auburn Hills, Mich. FIGS. 3a and 3b show schematics of the operating principle of a curved lens. Specifically, FIG. 3a illustrates x-ray diffraction from a crystal lattice according to Bragg's equation nλ=2d×sin θ, where λ is the wavelength, d is the separation of the crystal layer, n is an integer, and θ is the scattering angle with the crystal plane; FIG. 3b illustrates a curved multi-layer crystal lens with varying scattering angles (e.g., θ₁ and θ₂) and varying layer separations (e.g., d₁ and d₂) designed to bring diffracted x-rays from different planes of a focus. Depending on the material, a lens preferentially selects photons within a certain energy range from the incident spectrum, and focuses only these photons to a localized region in space.

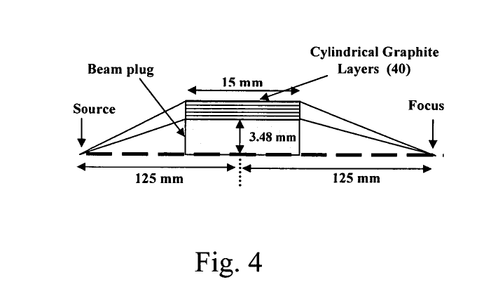

An example of a lens 40 suitable for use in the system 10 is shown as a schematic in FIG. 4. The multilayer graphite crystal lens 40 is about 15 mm long and about 2 mm thick. The diameter of the inner beam plug is ˜7 mm. The lens 40 is placed midway between the x-ray source and the focus, a distance of ˜25 cm. The lens 40 is specified to have a focus of about 1.0 mm FWHM for a 0.3 mm x-ray source.

By placing the lens 40 midway between the source and the focus, a uniform cylindrical multilayer graphite crystals can be used, which makes cutting simpler. The shorter “working condition” will also improve output and is desirable for dose painting.

This “pencil beam zone” is about 5 cm or longer. The lens replacement gain factor is about 3×. The lens 40 also reduces “spoke” intensity. The presence of “spokes”, however, is not of concern since they will be included in the dose calculation model.

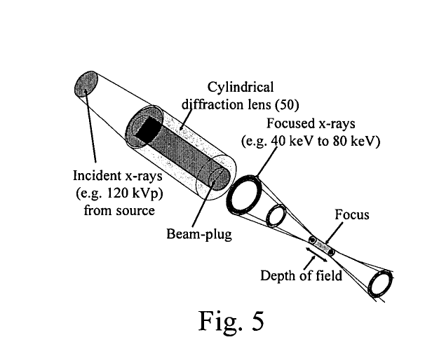

FIG. 5 shows a schematic view of an alternative curved graphite lens 50 constructed to focus x-rays with energies from 40 keV to 80 keV. The lens 50 is about 11 cm long, with an outer diameter of about 4 cm, and about a 2 cm wide central beam plug. The actual emitted spectral distribution depends on the input x-ray energy fluence distribution, and the variation in lens collection efficiency as a function of photon energy. For example, the lower collection efficiency at the higher x-ray energies can be offset by increasing the fluence of these photons in the input beam.

The entrance face of the lens 50 is at 17 cm from the x-ray source. X-rays are emitted in a conical shell to converge at the nominal focus 25 cm downstream from the emitting face of the lens 50. The shape and dimension of the focus and the depth of field are dependent on the lens design, and the size and shape of the x-ray source. The depth of field can be long due to the “mosiacity”, or irregularity, of the graphite crystal lattice, resulting in a pencil-like beam in the focal region.

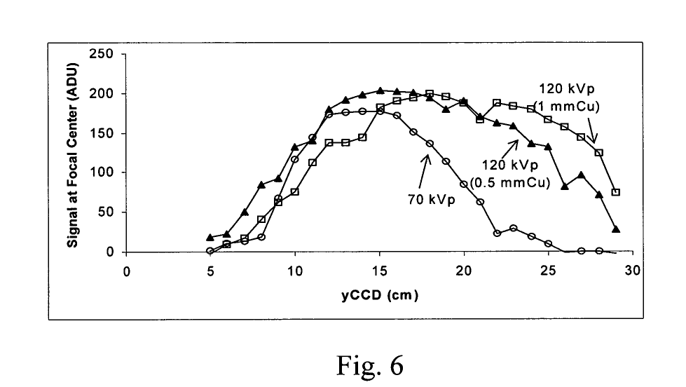

As an example, an x-ray source with a focal spot of 0.6 mm was used to produce three beams of different energies to incident on the lens; 70 kVp with no filtration, 120 kVp with 0.5 mm Cu filtration, and 120 kVp with 1.0 mm Cu filtration, respectively. The emitted focused beams, in air, had energies ranging from 40-80 keV as dictated by the lens. These beams exhibited pencil beam-like characteristics in the focal zone, or depth of field, where the beam intensities were relatively flat. FIG. 6 shows the intensities of the three beams along the central axis, yCCD, downstream from the emission face of the lens. The depth of field increased with increasing beam energy, and ranged from 5 cm to 8 cm.

Thus, a multi-layer curved lens can be used to focus 40-80 keV x-rays from a 100 kVp input beam to within a FWHM of ˜1 mm. Significant output gain can be achieved by using an x-ray lens for localize irradiation of small regions (<4 mm). The lens output can be increased by using a 250 kVp input beam, so as to increase the fluence in the 40 to 80 keV range.

Alternatively, two x-ray sources for irradiation can be employed. With the 250 kVp generator, the 2-source combination provides output at 120-150 cGy per min for large field irradiation; at 160-200 cGy per min for the focused irradiation. In a particular implementation, our source is a therapy source and another source is an imaging source. A flat panel detector is associated with each source.

The system 10 supports three dimensional (3D) treatment planning and accurate dose calculation to optimize beam arrangements for animal irradiation. Treatment planning employs high resolution helical CT, cone-beam CT, and images from other modalities. When appropriate, small animal MRI/MRSI resources can be used to improve target definition, and can be fused with the CT planning data.

The system 10 achieves dosimetric evaluation of about 5% to agreement (in relative dose) at the point of interest, and no worse than 10% (in relative dose) within 80% of the field boundary. A distance to agreement criterion of about 1 mm is adopted for those discrepancies that occur at high dose gradient regions. Comparison in terms of relative dose is chosen because substantial uncertainty (>10%) remains in absolute dosimetry using ionization standards.

To begin, an output factor at a reference position is established for dose prescription for both focused and “unfocused” broad beams. In order to minimize the effects of steep inverse square and finite size of the dosimeter, the output factor is defined as the dose in air on a central axis of a 10 cm×10 cm field at an extended SSD of 100 cm. Measurements are made in accordance to the recommendations by MPM Task Group 61 on low photon energy ionization dosimetry. The ionization standard is then used to calibrate water equivalent radiochromic films at different doses. These “secondary standard” film dosimetry is then used to determine relative dosimetry at the high spatial resolution required by experiments using the system 10.

Both the focused (40 to 80 keV) beam and the “unfocused” broad x-ray beam are modeled for dose calculations in the medium. Different strategies are adopted for the focused and broad x-ray beams because of the differences in their intended applications.

The focused “40-80 keV” beams is used to irradiate small (<4 mm) soft tissue volumes where bony perturbation effects are minor. The focused beam is treated as a pencil beam. It follows then that a dose calculation method that relies on measured data in homogeneous water-like medium is practical (and probably more accurate). This will account for all the complex attenuating and scattering effects that will alter the beam profiles in water. Two-dimensional radiochromic films, appropriately calibrated for dose output, is used as dosimeters. The method of Williamson is employed to ensure high resolution (at 0.1 mm), high quality (2% agreement with other standards) dosimetry measurements. Radiochromic films are placed between the horizontal slabs of a semi-infinite (half-air/half-medium) solid water slab phantom. (See, e.g., Meigooni, A. S., et al., Dosimetric characteristics of an improved radiochromic film. Med Phys, 1996. 23 (11): p. 1883-8 and Zhu, Y., et al., Quantitative evaluation of radiochromic film response for two-dimensional dosimetry. Med Phys, 1997. 24 (2): p. 223-31, the entire contents of which are incorporated herein by reference.) The focused beam is incident from the vertical direction. A range of “lens to surface distances” (LSDs) encompassing the useful pencil beam region is examined. For each LSD, film measurements are made at adequate number of depths to allow for proper interpolation of 3D pencil beam dosimetry. In addition, the measurements are repeated for focused beams incident from different oblique angles from vertical to horizontal to account for obliquity effect. A beam library of 3D isodose distributions as a function of LSD and incident angles are constructed. Dose delivered by multiple focused beams, from different incident LSDs and incident angles, are a summation of the appropriate beam data from the library.

The perturbation effect of irregular surface curvature is assumed to be negligible due to the small 1.0 to 1.5 mm FWHM dimension of the focused beam. In the event that inhomogeneity correction is necessary, a primary only model is adopted. This is facilitated by measuring the spectral distribution of the pencil beam in air along the pencil beam zone using a diagnostic x-ray beam spectral analyzer, such as produced, for example, by Amptek, Inc, Bedford, Mass. An inhomogeneity correction factor (ICF) is calculated according to the Eqn. 1, where hv denotes a specific photon energy bin; (D(hv) the photon fluence at energy bin hv,

$$ICF = \frac{ΣΦ ( hv ) {ⅇ}^{- Σμ ( hv , ρ , Z ) Δ x} hv \frac{μ abs}{ρ} ( hv , ρ , Z )}{ΣΦ ( hv ) {ⅇ}^{- Σμ ( hv , water ) Δ x} hv \frac{μ abs}{ρ} ( hv , water )} \qquad Eqn . 1$$

μ(hν, ρ, Z) the linear attenuation coefficient as a function of photon energy, electron densityp, and atomic number Z; Δx the incremental primary ray-path distance and μabs/σ (hμ, σ, Z) the mass energy absorption coefficient as a function of photon energy, electron density r, and atomic number Z. The combination of electron densityp and atomic number Z in the numerator is used to represent a specific material, as contrasts with water in the denominator. Eqn. 1 simply gives the ratio in dose in medium and in water respectively because of contributions from the different primary photons in the beam. The ICF is used to correct the data from the beam library. The ICF does not contain scatter information and therefore will not modify the beam profile.

For broad beam dose calculations, Monte Carlo methods provide a comprehensive approach to account for multiple scattering and bone attenuation effects. The EGSnrc code employed as it is well benchmarked for human radiation therapy applications and well maintained by the Ionization Standards Group at the National Research Council of Canada. The methodology for characterizing a therapy beam for dose calculation with EGSnrc or other Monte Carlo methods is well established. Its extension for the system 10, however, requires additional modeling efforts.

For the system 10, the model of the actual production of x-rays from the Comet dual focal sources is not performed. Instead, a semi-empirical approach of directly measuring the “phase-space”, or positional, distributions of emitted photon energy spectrum in air as input for Monte Carlo dose calculation is performed. The simplest approach and often effective approach, employs differential filtration measurements, coupled with an empirical formulae to determine the spectral distributions of the kV beams.

Such spectral information has been incorporated for Monte Carlo calculations that agree well (˜3%) with point dose measurements made with detectors of different compositions. The “phase-space” information may have minimal spatial variation for small field sizes of the order of 50 mm×50 mm for SSD at ˜25 cm.

When the “phase-space” varies across the beam area, thereby affecting off-axis dose calculation, the direct approach of measuring the information with a spectral analyzer is performed. The detector face of the spectral analyzer is collimated with a, ˜0.1 to 0.2 mm, Tungsten collimator disk and scanned to measure the energy distributions across the beam area at distances of 20 cm, 25 cm and 30 cm from the source, i.e., on either side of the intended isocenter of the system 10. The dimension of the Tungsten collimation is chosen to be compatible with the calculation resolution. Care is taken to account for varying obliquity incidence as the detector is scanned across the beam. The sampling points are distributed to assure adequate data collection within reasonable time limits. For the broad x-ray beams, the phase-space distributions measurements are initially made for field sizes of 3 mm×3 mm, 10 mm×10 mm, and 30 mm×30 mm, defined at 25 cm from the source.

It is assumed that the energy distributions from the source are due to primary photons only. The primary photon model by comparing extrapolated “phase-space” data to measurements at different field sizes and distances from the source. If valid, only large field measurements are needed to commission the phase-space data. If not, field size dependence data need to be measured using a larger number of field sizes.

Incorporating the measured phase-space energy fluence information into EGSnrc transport code is sufficient for accurate kV dose calculation in homogeneous media. The EGSnrc calculations are validated with several measurements. The calculated spectral changes due to interspersing slab phantoms downstream from the source are validated with a spectral analyzer. Dose distributions in the depth and cross-beam directions are measured in water-like medium with radiochromic films and compared with EGSnrc calculations. Further improvement in fitting can be made by convolving the Monte Carlo results with a penumbral spreading function to reflect blurring contribution from the finite source size. Similar approach is used in commissioning a photon pencil beam algorithm for radiation therapy. For a homogeneous water medium, a 5% agreement in relative dose distribution within 80% of the field boundary is achievable between EGSnrc and measurements. In other high dose gradient regions, a 1 mm distance to agreement should be attainable.

The spectral distributions of the x-rays may not vary significantly for field size less than 50 mm×50 mm, a pencil beam summation method may also be highly effective for very fast dose calculations. An elemental pencil beam in water (at 0.2 mm voxel resolution) can be generated by EGSnrc using the measured phase space spectral distributions. This elemental pencil includes the contributions from multiple scatter. Broad beam dosimetry is then simply the summation of contributions from many pencil beams. Again, a penumbral spreading function can be tuned to improve agreement with measured data. Once commissioned, the elemental pencil beam and the penumbral function can be used to calculate doses for arbitrary beam shaping and radiation geometry. A primary inhomogeneity correction method, identical to that of Eqn. 1, can be used to correct each pencil beam. Pencil beam calculations are fast, although, they do not account for scatter changes or loss of electronic equilibrium due to inhomogeneity effects. However, the resultant perturbations of the dose distribution may be local, and may not be of concern. Such a method of very fast calculation is highly desirable as it enhances our planning capability to explore different irradiation options.

Focused beam dose distributions are calculated at a 0.1 mm voxel resolution in a grid 60×60×800 dose points for the largest 4 mm×4 mm field to a depth of 8 cm, allowing for beam spreading at the edges. The range of depth may be changed depending on dimension of “pencil beam zone”. The broad beam distributions are calculated at 0.2 mm voxel resolution, depending on the irradiation experiments and algorithms. The dose grid is of the order of 250×250×400 points. About an hour is needed for the lengthier Monte Carlo dose calculations using, for example, a 4-processor PC.

For both the focused and broad beam beams, validation is performed using a set of phantoms and irradiation geometries commonly employed for evaluating dose calculations. Phantoms contain materials that are water-, lung- and bone-like, of well-defined compositions. They will also have idealized shapes, such as cylinders, that can be simulated, or scanned by helical CT or cone-beam CT system as input for dose calculation. There may be (minor) variation in CT numbers that are imaging technique dependent. These validation exercises help establish proper conversion of CT numbers to appropriate x-ray interaction coefficients.

Different beam arrangements are considered, including shaped fields with or without physical compensators, variable SSDs and oblique incidences. 2D measurements are made using radiochromic films and compared with calculations. For 3D validation, experiments involving multiple beams from different gantry angles, such as those in radiosurgery, are used to irradiate a 3D gel dosimetry system. The gel dosimetry is analyzed using an optical CT system.

Thus, there are 2 modes of irradiation using the system 10. When the target volumes are <4 mm diameter, the 40-80 keV lens is deployed. For larger field dimension, physical collimation can be used for beam shaping as it does not shield off part of the x-ray source and reduce output. X-rays up to 250 kVp can be used. For both modes of operation, a 3D treatment planning and dose calculation system for the kVp x-rays is desirable.

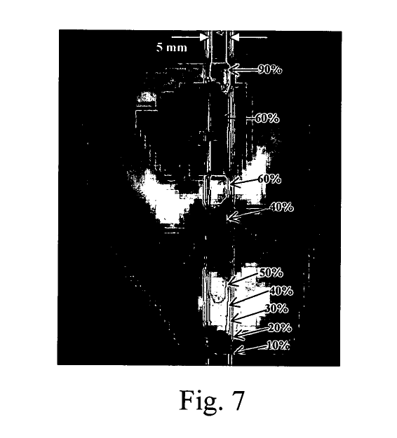

FIG. 7 shows the relative isodose distributions in an irradiated rabbit's head. The beam entered from the rear of the tilted rabbit's head. Dose enhancement at the bony structures was observed, but did not greatly restrict the depth penetration of the 120 keV x-rays. Use of higher energy, i.e. filtered, x-rays for radiation experiment with larger animals is desirable in order to minimize the bone-enhanced attenuation of the beam. The pencil beams are suitable for irradiation of smaller animals with minimal bony structures along the beam path; avoiding the large bone attenuation of the focused 40 to 80 keV x-rays.

FIG. 7 demonstrates the principle of coupling CT-based EGSnrc calculations with a 3D treatment planning system to provide planning capabilities for the system 10. It is interesting to note that the beam edges remained sharp for the 5 mm by 5 mm field size as it traversed through the rabbit's head. The observation suggests that for a very small beam, such as “pencil beam” accurate dose calculations can be achieved by using a measured dose spread kernel in a pencil beam convolution model as an alternative to Monte Carlo methods. A hybrid approach may be used in which the fast planning calculation of the pencil beam model is augmented with a final Monte Carlo calculation.

The system 10 achieves stability and reproducibility of animal setup for single or multiple fractionated irradiations. The system 10 can achieve setup margin of 2 mm or less for repeat broad beam irradiation, and even smaller to complement the highly localized irradiation using focused x-rays. Achieving these margin specifications may be used in conjunction with immobilization, in combination with on-line radiographic or tomographic image guidance for positioning. Radiographic guidance based on bony landmark allows for more efficient setup, thus throughput, of fractionated irradiation.

Immobilization methods for different anatomical regions of the animals include vacuumed foam bags or suctioned sheets. Animal immobilization may employ general anesthesia for certain procedures. For example, rabbits can be anesthetized with ketamine-xylazine, while rats and mice can be anesthetized using ketamine-acepromazine. Combinational methods of sedation, hooded ventilation or even invasive ventilation can be applied for effective motion immobilization.

The system 10 can use multi-modality information to define a target for accurate irradiation that are based on functional or biological imaging information. Determination of margins allow for the incorporation of setup uncertainty in outcome analysis. Various general methods can be employed to obtain the margins. For example, for each irradiation procedure, a “before and after” cone-beam CT is acquired of the animal to determine how much the animal might have moved during the procedure, or the flat-panel detector can be used for acquiring portal images from its opposed upstream x-ray source. Film attachments are implemented to capture the portal images of the other two beams that do not have complementary flat panel detectors. These portal images are back-projected through the CT volumes to determine the accuracy and precision with which the beams were able to “hit” the target.

Lastly, in vivo dosimetry validation may be performed on the system 10, particularly for localized irradiation using focused beams where transmission intensities might be too low to form a portal image. For focused irradiation, delivery of dose to TLD dosimeters into cavities of mice and rats may be performed. CT scans are acquired of the animals with the TLDs already inserted. The use of TLD for in vivo dose verification is quite adequate because the effects of its non-water-like composition can be accounted for accurately using the EGSnrc Monte Carlo calculations which employs only in-air photon “phase-space” information. A correction factor for the energy absorption coefficients can also be applied if the pencil beam model is used for calculation. The TLDs can also be pre-calibrated by irradiation with the system 10.

Other embodiments are within the scope of the following claims.

What is claimed is:

1. A system for radiation imaging and therapy of small animals comprising:
a gantry with an aperture, the gantry being isocentric and rotatable about its center;
at least one radiation source mounted to the gantry, the radiation source being a constant potential x-ray source with a shutter and moving along an arcuate path as the gantry rotates, the radiation source emitting an x-ray beam at a first energy level for imaging of a small animal and emitting an x-ray beam at a second energy level for radiation therapy of the small animal, the first energy level being less than the second energy level; and
a flat panel detector that receives x-ray beams after they pass through the small animal.

2. The system of claim 1 further comprising one or more additional radiation sources mounted to the gantry, each of the one or more radiation sources being a constant potential x-ray source with a shutter moving along the arcuate path and emitting an x-ray beam at an energy level greater than the first energy level.

3. The system of claim 2 wherein each of the additional radiation sources emits an x-ray beam at substantially the second energy level.

4. The system of claim 2 wherein the angular separations between the radiation sources about the center of the gantry are substantially equal.

5. The system of claim 2 wherein the radiation sources provide a combined radiation dose rate of about 200 cGy/min.

6. The system of claim 1 wherein the radiation source emits x-ray with an energy level in the range between about 100 and 250 kVp.

7. The system of claim 1 wherein the first energy level is about 100 kVp.

8. The system of claim 1 wherein the second energy level is about 250 kVp.

9. The system of claim 1 further comprising an x-ray lens coupled to the radiation source for focusing the x-ray beam.

10. The system of claim 9 wherein the energy level of the focused x-ray beam is in the range between about 40 and 80 keV.

11. The system of claim 9 wherein the imaging is cone-beam CT imaging.

12. The system of claim 9 wherein the x-ray lens is a multilayer crystal lens.

13. The system of claim 12 wherein the x-ray lens is a curved multilayer crystal lens.

14. The system of claim 12 wherein the x-ray lens is a cylindrical multilayer crystal lens.

15. The system of claim 1 further comprising a couch upon which the small animal is positioned, the couch being movable through the aperture.

16. The system of claim 1 wherein the arcuate path is a circular path.

17. The system of claim 1 wherein the flat panel detector is an a:Si flat panel detector.

18. A method for radiation imaging and therapy of small animals comprising:
positioning the small animal in an aperture of a gantry;
rotating one or more radiation sources mounted to the gantry along an arcuate path about the small animal, each radiation source being a constant potential x-ray source with a shutter, one of the radiation sources emitting an x-ray beam at a first energy level for imaging of the small animal and emitting an x-ray beam at a second energy level for radiation therapy of the small animal, the first energy level being less than the second energy level; and
detecting x-ray beams passing through the small animal with a flat panel detector.

19. The method of claim 18 wherein the other radiation sources emit an x-ray beam at substantially the second energy level.

20. The method of claim 18 further comprising focusing the x-ray beams with x-ray lens coupled to respective radiation sources.
